(19) United States
(12) Patent Application Publication
(10) Pub. No.: US 2008/0103503 A1
(43) Pub. Date: May 1, 2008

(54) Fastener System of the Captive Screw Type

(75) Inventors: Didier Roux, Asnieres-sur-Oise (FR); Xavier Renard, Vauhallan (FR)

Correspondence Address: YOUNG & THOMPSON, 209 Madison Street, Suite 500, ALEXANDRIA, VA, 22314, US

(73) Assignee: DIDIER ROUX, ASNIERES-SUR-OISE (FR)

(21) Appl. No.: 11/795,290

(22) PCT Filed: Dec. 29, 2005
(86) PCT No.: PCT/FR05/03297

§ 371 Date: Jul. 16, 2007

(30) Foreign Application Priority Data
Jan. 17, 2005 (FR) 0500444

(51) Int. Cl. A61B 17/80; A61B 17/86

(52) U.S. Cl. 606/69; 606/73

(57) ABSTRACT

A screw fastener system includes a plate having formed therein, centered on an axis perpendicular to the plane of the plate: a circularly generally cylindrical orifice of depth h and of cross-section equal to D, an orifice of cross-section equal to d less than D, and a housing, the orifices being situated on either side of the housing, the height of the housing being equal to H and its width having a value L not less than D, and a fastener screw constituted by a shank carrying a thread, the section S of the shank being no greater than the value d, and a head arranged for screwing into the cylindrical orifice, the height E of the head being no greater than the height H of the housing. The system is applicable to fastening implants such as spinal or other osteosynthesis plates.

The present invention relates to screw fastener systems of the type having a captive screw and having a particularly advantageous, but non-exclusive, application in fastening surgical implants such as an acetabulum, or an osteosynthesis plate, etc.

Fastener systems are known for fastening on a body of any kind by screwing in a screw, with a washer or any other element, such as a connection plate or the like, being interposed between the head of the screw and the body into which the threaded shank of the screw is to be screwed.

Such fastener systems raise essentially two types of problem. One of the problems is that of keeping the screw in place together with the plate or the washer while the screw is being screwed in. The other is that of the reliability of the screw fastening over time, i.e. the risk of the screw coming unscrewed for any reason whatsoever, for example under the action of vibration or the like, and even the risk that the screw might become completely detached from the body and lost.

This second problem is particularly troublesome when installing a spinal or other osteosynthesis plate, since unscrewing of the screw or screws can be very dangerous for the patient in whose body the screws have been implanted to hold the plate in place.

Numerous devices already exist that enable screw fastening to be performed using screws of the captive type, e.g. that which is described and shown in US-A-2004/260306. Nevertheless, all such devices are relatively complex, and therefore expensive.

An object of the present invention is thus to provide a screw fastener system that mitigates the above-mentioned drawbacks of prior art devices, while being very simple in structure, and easy to use.

More precisely, the present invention provides a screw fastener system, characterized in that it comprises:

firstly a plate having formed therein, substantially centered on a common “fastener” axis substantially perpendicular to the plane of said plate, the following:
a first orifice of generally circularly cylindrical shape of depth h and of cross-section equal to D;
a second orifice of cross-section equal to d less than D; and
a clearance housing, said first and second orifices being situated on either side of said clearance housing and opening out therein, the height of said clearance housing along said fastener axis being equal to H, and its width measured perpendicularly to said fastener axis having a value L not less than D; and

secondly a fastener screw constituted by a shank carrying a thread, the cross-section S of said shank, perpendicular to its axis, being no greater than the value d, and a shouldered head secured to one end of said shank, said shouldered head having a peripheral portion arranged to be suitable for screwing into a side portion (13) of the first orifice (10) bordered by the wall (11) of said first orifice, the height E of said shouldered head taken along the axis of the shank being no greater than the height H of the clearance housing.

Other characteristics and advantages of the invention appear from the following description given with reference to the accompanying drawings by way of non-limiting illustration, in which:

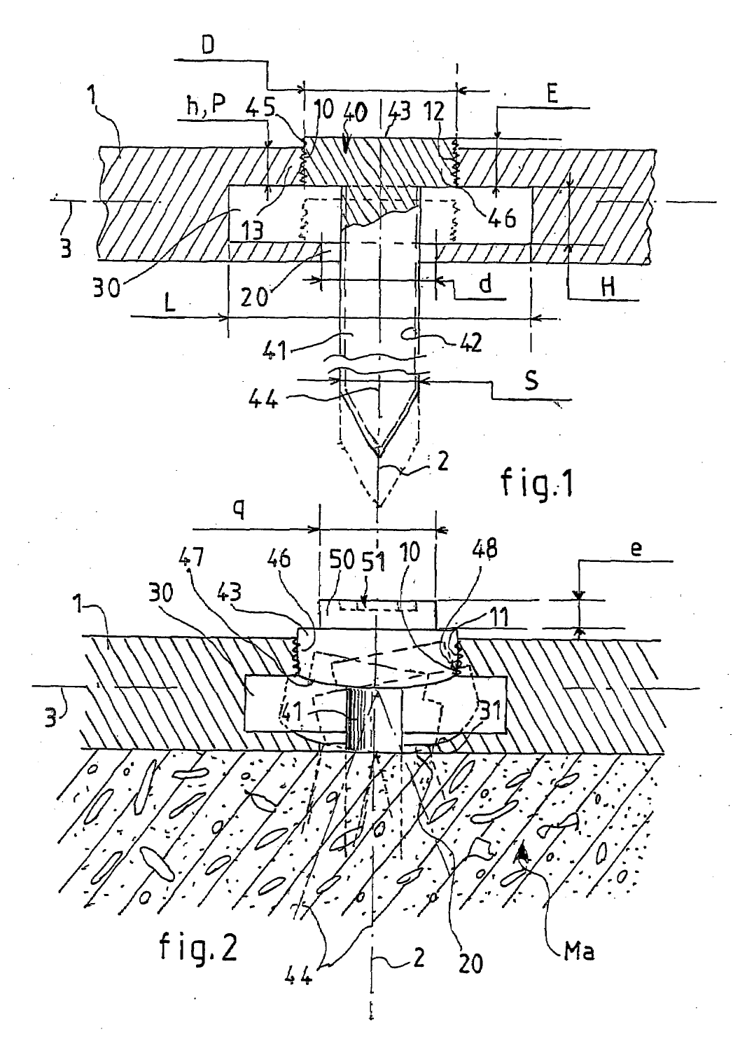

FIGS. 1 to 3 show three respective embodiments of screw fastener systems of the invention.

With reference to accompanying FIGS. 1 to 3, the screw fastener system comprises a plate 1 having, substantially centered on a common “fastener” axis 2 that is substantially perpendicular to the plane 3 of the plate, the following: a first orifice 10 of generally circular cylindrical shape of depth h and of cross-section equal to D; a second orifice 20 of cross-section equal to d, less than D; and a clearance housing 30; the first and second orifices 10, 20 being situated on either side of the clearance housing 30 and opening out therein. The height of the clearance housing 30 along the fastener axis 2 is equal to H, and its width taken perpendicularly to said fastener axis has a value L that is not less than D.

The system also includes a fastener screw 40 constituted by a shank 41 carrying a thread 42, the cross-section S of the shank, perpendicular to its axis 44, being no greater than the value d, and a head 43 presenting a shoulder and secured to one end of the shank, the shouldered head having a peripheral portion 46 arranged to be suitable for screwing into the side portion 13 of the first orifice 10 surrounded by the wall 11 of said orifice, the height E of the shouldered head along the axis 44 of the shank 41 being no greater than the height H of the clearance housing 30.

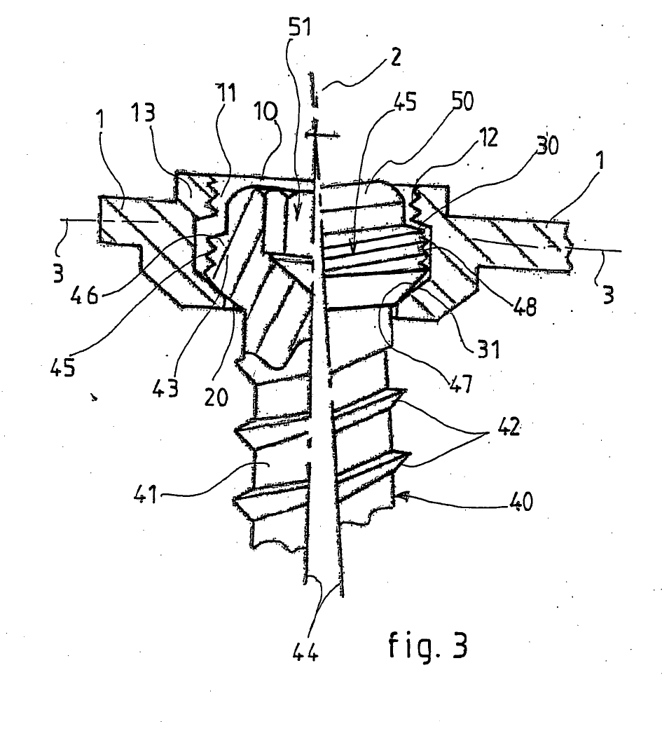

In a first embodiment, as shown in FIGS. 1 and 3, the wall 11 of the first orifice 10 is tapped 12 over a length P, and the peripheral portion 46 of the shouldered head 43 carries a thread 45 complementary to the tapping 12.

Nevertheless, in another possible embodiment, as shown more particularly in FIG. 2, the peripheral portion 46 of the shouldered head 43 and the side portion 13 of the first orifice 10 are made respectively of first and second materials presenting different hardnesses, one of the two elements constituted by the “peripheral portion 46 of the shouldered head 43” and by the “side portion 13 of the first orifice 10 having the tapping 12” being made of the harder material, with the other element being suitable for screwing therein with self-tapping.

By way of example, one of the two materials is T40, i.e. pure titanium, while the other is TA6V, i.e. a titanium alloy.

It is specified that the term “tapping” as used above covers both tapping proper for the side portion 13 of the first orifice 10, and cutting a thread on the peripheral portion 46 of the shouldered head 43.

It is specified that the side portion 13 of the first orifice 10 and the peripheral portion 46 of the shouldered head 43 may be constituted respectively by a lining on the wall 11 of the orifice 10, and/or by a sleeve situated on the periphery of the shouldered head 43, with the remaining portions of the plate 1 and of the screw 40 possibly being made of the same material.

Under such circumstances, it is even preferable for the peripheral portion 46 of the shouldered head 43 to be made of a sleeve of a material such as T40, while the entire plate 1 is made of a softer material, such as TA6V. It is the wall 11 of the orifice 10 that will be tapped 12, i.e. it is the shouldered head that will screw into the tapping 12 by self-tapping, as in the embodiment shown diagrammatically in FIG. 2.

In an embodiment shown in all the figures, the value d is greater than the value S. Under such circumstances, as shown more particularly in FIGS. 2 and 3, the shank 41 of the screw may occupy different angular positions relative to the plane 3 of the plate 1, at least within limits that are generally sufficient for putting osteosynthesis plates into position, in particular.

In order to facilitate orienting the shank of the screw in this variety of angular positions, and in order to give the shouldered head a good seat, the wall portion 31 of the clearance housing 30 surrounding the opening through which the second orifice 20 opens out into said clearance housing presents a concave shape, and the portion 47 of the side surface of the shouldered head 43 connecting the shank 41 to the portion 48 of said side surface that is suitable for screw fastening presents a convex shape that is substantially complementary to the concave shape. These complementary concave and convex shapes are advantageously hemispherical or the like.

When the plate 1 is constituted by a spinal osteosynthesis plate for interconnecting two vertebrae, each fastener screw has a shank 41 threaded with a self-tapping bone thread.

In an embodiment that can be advantageous, in particular for making it easier to manipulate the fastener screw so as to implant it in a material such as bone or the like, see FIGS. 2 and 3, the system also has a stud 50 surmounting the shouldered head 43, with the cross-section g of the stud, taken perpendicularly to the axis 44 of the shank 41, being less than the value D.

In an application to installing an osteosynthesis plate or the like, in order to ensure that the stud 50 does not project beyond the plate 1 when the screw 41 is screwed into a bone, the sum of the height E of the shouldered head 43 plus the height e of the stud 50 (FIG. 2) is no greater than the sum of the height H of the clearance housing 30 plus the depth h of the first orifice 10.

It should be observed that this depth h of the first orifice 10 and the height P of the tapping 12 need not be equal, for example as happens when the orifice 10 is extended by a leading countersunk hole of frustoconical or cylindrical shape of diameter greater than D. Nevertheless, in a preferred embodiment such as the embodiment shown, these two values h and P are equal.

For an application to installing a spinal or other osteosynthesis plate, it is advantageous for the pitch of the tapping 12 of the first orifice 10 to be no greater than the pitch of the thread 42 on the threaded shank 41, and preferably for them to be equal.

1. A screw fastener system, characterized in that it comprises:
firstly a plate (1) having formed therein, substantially centered on a common “fastener” axis (2) substantially perpendicular to the plane (3) of said plate, the following:
a first orifice (10) of generally circularly cylindrical shape of depth h and of cross-section equal to D;
a second orifice (20) of cross-section equal to d less than D; and
a clearance housing (30), said first and second orifices (10, 20) being situated on either side of said clearance housing (30) and opening out therein, the height of said clearance housing (30) along said fastener axis (2) being equal to H, and its width measured perpendicularly to said fastener axis having a value L not less than D; and
secondly a fastener screw (40) constituted by a shank (41) carrying a thread (42), the cross-section S of said shank, perpendicular to its axis (41), being no greater than the value d, and a shouldered head (43) secured to one end of said shank, said shouldered head having a peripheral portion (46) arranged to be suitable for screwing into a side portion (13) of the first orifice (10) bordered by the wall (11) of said first orifice, the height E of said shouldered head taken along the axis (44) of the shank (41) being no greater than the height H of the clearance housing (30).

2. A fastener system according to claim 1, characterized by the facts that the wall (11) of the first orifice (10) includes tapping (12) over a length P, and that the peripheral portion (46) of said shouldered head (43) includes a thread (45) complementary to said tapping (12).

3. A fastener system according to claim 1, characterized by the facts that the peripheral portion (46) of the shouldered head (43) is made of a first material, and that the side portion (13) of said first orifice (10) is made of a second material, the first and second materials having different hardnesses, one of the following two elements constituted by the “peripheral portion (46) of the shouldered head (43)” and by the “side portion (13) of said first orifice (10) having tapping 12” being made of the harder material, the other element being suitable for self-tapping screw engagement therewith.

4. A fastener system according to claim 3, characterized by the fact that one of the first and second materials is T40 (pure titanium) and the other of the two materials is TA6V (titanium alloy).

5. A fastener system according to claim 1, characterized by the fact that the value d is greater than the value S.

6. A fastener system according to claim 1, characterized by the fact that said threaded shank (41) has a self-tapping thread.

7. A fastener system according to claim 1, characterized by the fact that it includes a stud (50) surmounting said shouldered head (43), the cross-section g of said stud, taken perpendicularly to the axis (44) of said shank (41), being less than the value D.

8. A fastener system according to claim 7, characterized by the fact that the sum of the height E of said shouldered head (43) plus the height e of said stud (50) is no greater than the sum of the height H of the clearance housing (30) plus the depth h of said first orifice (10).

9. A fastener system according to claim 2, characterized by the fact that the pitch of the tapping (12) is no greater than that of the thread (42) of the threaded shank (41).

10. A fastener system according to claim 1, characterized by the facts that the wall portion (31) of said clearance housing (30) bordering the opening whereby said second orifice (20) opens out into said clearance housing presents a concave shape, and that the portion (47) of the side surface of said shouldered head (43) connecting the shank (41) to the portion (48) of said side surface that is suitable for screw fastening presents a convex shape that is substantially complementary to said concave shape.

11. A fastener system according to claim 2, characterized by the fact that the value d is greater than the value S.

12. A fastener system according to claim 2, characterized by the fact that said threaded shank (41) has a self-tapping thread.

13. A fastener system according to claim 2, characterized by the fact that it includes a stud (50) surmounting said shouldered head (43), the cross-section g of said stud, taken perpendicularly to the axis (44) of said shank (41), being less than the value D.
